(19) United States
(12) Patent Application Publication
(10) Pub. No.: US 2011/0196356 A1
(43) Pub. Date: Aug. 11, 2011

(54) ABLATIVE/COAGULATIVE UROLOGICAL TREATMENT DEVICE AND METHOD

(75) Inventor: Wolfgang Neuberger, Dubai (AE)

(73) Assignee: CeramOptec Industries Inc.

(21) Appl. No.: 12/882,758

(22) Filed: Sep. 15, 2010

Related U.S. Application Data
(60) Provisional application No. 61/242,677, filed on Sep. 15, 2009.

(51) Int. Cl. A61B 18/22

(52) U.S. Cl. 606/15

(57) ABSTRACT

A device/system and a method of treating enlarged prostate and other urologic abnormalities are presented. A combined treatment is performed with several interstitial coagulating probes and an ablating fiber. Tissue vaporization is minimized by the denaturalizing effect of interstitial coagulative fibers. In a single device, multiple delivery systems achieve optimal tissue ablation/coagulation; a non-laser source like microwave energy coagulates tissue and a laser source ablates tissue. Another device comprises two or more laser sources with adjustable wavelengths controllable by physician as to ablative, coagulative, and tissue penetration needs. Continuous, semi-continuous, pulsed wave, or combinations are useful. In another embodiment, optical fiber has a central core for transmitting laser radiation, and a cladding layer about the core that may further transmit other laser radiation of a different or a same wavelength as the core. Fibers may have a side-firing distal end, a radial firing end, or an off-axis firing end. Feedback controls can be used. In general coagulative irradiation can use a radiofrequency or other radiant thermal source.

### DOMESTIC PRIORITY UNDER 35 USC 119(e)

This application claims the benefit and priority of U.S. Provisional Application Ser. No. 61/242,677 filed Sep. 15, 2009, entitled “Ablative/Coagulative Urological Treatment Method and Device” by Wolfgang Neuberger, which is incorporated by reference herein.

### BACKGROUND OF THE INVENTION

1. Field of the Invention

The present invention is related to minimally invasive devices for treating Benign Prostate Hyperplasia (BPH). More particularly the invention relates to simultaneous prostate ablation and coagulation devices and methods for BPH treatment.

2. Background Disclosure Statement

Numerous approaches have been developed for treating BPH. The desired objective is to eliminate prostate obstruction of the urethra effectively and minimizing recurrence, bleeding, damage to adjacent tissue, side effects and patient pain and discomfort among other things. This has been done using different methods.

In some cases, medications such a-blockers and anti-androgens can be used to treat BPH. However in men with severe symptoms, these are only palliative and have unwanted side effects that sometimes arise years after treatment. Alpha-blockers do not modify prostate growth, and even the use of prostatic growth inhibitors such as finasteride (Proscar) or dutasteride (Avodart) often fails to prevent recurrent urinary symptoms of BPH and retention.

Transurethral resection of the prostate (TURP) (U.S. Pat. No. 6,156,049 by Lovato et al), consists of inserting a transurethral incisional device through the patient's urethra, incising off at least one piece of targeted prostatic tissue using the incisional device, inserting a morcellation probe through the patient's urethra, morcellating the excised piece of targeted prostatic tissue with the morcellation probe, and aspirating the morcellated prostatic tissue through the morcellation probe and out of the patient. This method is effective but it's known to cause numerous side effects, including incontinence, impotence, retrograde ejaculation, prolonged bleeding and TURP syndrome.

Photoselective vaporization of the prostate (U.S. Pat. No. 6,986,764 by Davenport et al) involves transmitting laser radiation with specific average irradiance to the treatment area, to form a spot of preset size. It uses a high-power potassium-titanyl-phosphate (KTP) laser, also called the “greenlight” laser. The delivered laser radiation has a wavelength between 200 nm and about 650 nm, and has an average irradiance in the treatment area greater than about 10 kilowatts/cm², in a spot size of at least 0.05 mm².

In transurethral microwave thermal therapy (TUMT) (U.S. Pat. No. 6,944,504 by Arndt et al), a Foley-type catheter containing a microwave antenna is placed within the urethra. The microwave antenna is positioned adjacent to the transitional zone of the prostate, where BPH occurs, and allows selective heating of the prostate. Maintaining the temperature of the BPH tissue above 45° C. leads to necrosis of the tissues and subsequent reabsorption of necrotic tissue by the body.

Holmium enucleation (HoLEP) of the prostate uses holmium laser energy to carve out the two lateral lobes of the prostate in an endoscopic version of an open enucleation. The tissue removed is generally too large to be removed through the resectoscope; therefore, a tissue morcellator must be introduced and the tissue, floating free in the bladder, must be captured and fragmented, while avoiding contact between the morcellator and the bladder wall. This method offers good hemostasis and allows tissue to be conserved for histological evaluation. However, this modality is technically challenging and can be quite time-consuming. The efficacy of the HoLEP procedure depends upon maintaining very close contact between the fiber and the tissue to be removed. As a result, it is possible to perforate the prostate during the procedure and many surgeons avoid it because of the difficulty in learning and maintaining proficiency in the technique.

Another non-invasive technique is transurethral needle ablation (TUNA). TUNA uses low level radiofrequency (RF) energy to heat the prostate. Using TUNA, two separate needles are inserted into prostate through the urethra. Several watts of RF energy are applied to each needle to cause thermal necrosis of the prostate cells around the needles. Application of this treatment to several sites of the prostate typically results in sufficient necrosis to relieve symptoms of the BPH.

These treatment approaches are either ablative or coagulative or try to minimize coagulation. Especially for an office procedure these methods have their drawbacks: while it is possible to treat even larger prostates (up to 100 g) with the ablative approach, the amount of intervention time required to eliminate large amounts of tissue can result in strain and stress on the patient who is usually fully conscious during the intervention. The extensive period of time required is also a cost factor for the operating urologist. Purely coagulative procedures, such as microwave or interstitial laser treatment are less stressful on the patient and once the interstitial fibers or electrodes have been placed, require less attention and time for the urologist. However, the clinical outcome is less certain and not guaranteed and occurs only after a delay of several weeks. As a consequence, immediate relief of symptoms is not is achieved when using purely coagulative procedures. Also, recurrences sometimes arise.

U.S. Patent Publication 2007/0219601A by Neuberger achieves tissue ablation as well as tissue coagulation substantially simultaneously by utilizing at least two wavelengths of light. The device and method improve urinary flow and minimize post-treatment blood loss and edema while maintaining a nearly blood-free operating field during treatment by irradiating with a combination of at least two different wavelengths of light. Tissue ablation is affected by having one wavelength that is highly absorbed in the prostatic tissue while another less highly absorbed wavelength coagulates surrounding tissues while maintaining minimal thermal damage to surrounding tissue. This procedure uses laser technology for both ablation and coagulation objectives. However many skilled in the art prefer other coagulation approaches such as microwave or interstitial laser treatment. Furthermore other newer and better coagulation methods may arise in time.

There is therefore a need for a combined treatment system that improves on the state of the art by allowing more precise and effective ablation and coagulation of abnormal soft tissue such as cancerous or hyperplasic prostate tissue. The present invention addresses this need.

### OBJECTIVES AND BRIEF SUMMARY OF THE INVENTION

It is an objective of the present invention to provide an improved minimally invasive device and method for treating Benign Prostate Hyperplasia, wherein a device and method provided to allow for performance of coagulative as well as ablative BPH treatment in one session by using two types of delivery systems embedded: a coagulative system, which is essentially placed and left in place during the necessary time to achieve sufficient coagulation; and an ablative system, that can be moved and manipulated by the surgeon during the treatment to assure tissue removal in the critical locations providing fast symptom relief.

It is also an objective of the present invention to provide a device and method for effective BPH treatment in an office setting that can minimize procedure duration, patient discomfort and recurrence of symptoms and complications.

It is still an objective to provide a system for BPH treatment that can take advantage of the benefits of coagulative and ablative procedures in one device.

It is yet another objective of the invention to provide a single device capable of administering two different types of energy through two corresponding probes or sets of probes for performing ablation and coagulation procedures substantially simultaneously.

It is a further objective of the present invention to provide an effective underskin prostate treatment which utilizes a control mechanism that delivers a predetermined energy to the prostate based on manual movement speed of the fiber under the skin and the prostate's physical parameters.

Briefly stated, a device/system and a method for the treatment of enlarged prostate and other urologic abnormalities are presented. This system enables the simultaneous attachment of several interstitial coagulative treatment probes as well as an ablative fiber to perform a combined treatment utilizing the intervention time and the time of the localized anesthesia effect in an optimal manner. The amount of tissue removed by the urologist by vaporization can be kept to a minimum, thanks to the (delayed) improvement of the achieved symptom scores resulting from the denaturalizing effect of the interstitial coagulative fibers. In one preferred embodiment, two or more types of delivery systems are embedded in a single device for achieving optimal tissue ablation and coagulation effects including at least one non-laser source such as microwave energy, capable of producing radiation energy to coagulate tissue and at least one laser source capable of producing radiation to ablate tissue. In another preferred embodiment, device comprises two or more laser sources which emit at adjustable wavelengths controllable by physician according to ablative and coagulative needs and tissue penetration needs depending on their effective absorption in different tissue components. Wavelengths ranges are chosen such that tissue absorption properties change sensibly with small variations of such wavelengths, based on a steep region of the absorption curve. Radiation may be applied in continuous, semi-continuous or pulsed wave, in different combinations. In another preferred embodiment, optical fiber has a central core for transmitting laser radiation, and a cladding layer about the core that may further transmit other laser radiation of a different or a same wavelength as the core. Fibers used in various embodiments may be, but are not limited to those comprising a side-firing distal end, a radial firing end, or an off-axis firing end. In a preferred embodiment, device includes a control mechanism which allows for the delivery of constant power density based on feedback regarding speed of fiber movement and local structural tissue parameters. In various embodiments, the coagulative irradiation can be done by a radiofrequency or other radiant thermal source.

The above and other objects, features and advantages of the present invention will become apparent from the following description read in conjunction with the accompanying drawings, (in which like reference numbers in different drawings designate the same elements).

### BRIEF DESCRIPTION OF FIGURES

FIG. 1 shows a plan view of a preferred embodiment whereby laser energy is used for ablation and microwave (MW) energy (or radiofrequency RF) is used for coagulation.

FIG. 2 shows a plan view of preferred embodiment whereby device delivers adjustable laser wavelengths.

FIG. 3a shows a plan view of preferred embodiment whereby device has two or more energy sources that deliver ablative and coagulative radiation to tissue through a unique optical fiber.

FIG. 3b shows a detailed view of fiber tip whereby optical fiber is composed of a core and two concentric claddings.

FIG. 3c shows a detailed view of another embodiment in which laser fiber has an off-axis firing end.

FIG. 4 shows a plan view of preferred embodiment whereby device includes a control mechanism which allows for the delivery of constant power density based on feedback regarding speed of fiber movement and local structural tissue parameters.

### DETAILED DESCRIPTION OF PREFERRED EMBODIMENTS

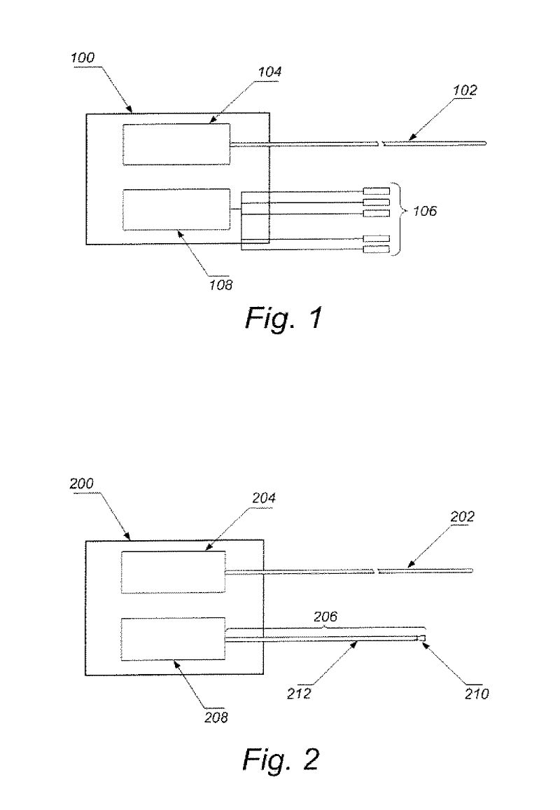

One preferred embodiment is shown in FIG. 1. The urological treatment device 100 consists of two energy sources. Laser energy source 104 drives optical fiber 102 for transurethral laser ablation treatment of the enlarged prostate. Its object is to vaporize a portion of the prostate tissue so as to allow for urethral flow. Laser energy is emitted at a controllable wavelength which may be adjusted by user between predefined ranges. In a preferred embodiment, laser radiation comprised between 1900 and 2000 nm is emitted. Mentioned wavelength range comprises a peak in water absorption at about 1950 nm and relatively very low absorption in other components present in tissue. Therefore, small variations in wavelength, within this small range modify tissue absorption behavior substantially. Physician can profit from this feature by varying emitting wavelength according to desired ablation effects. Microwave source 108 feeds a predefined number of microwave fibers, in the form of catheter array 106, for interstitial access to the prostate for coagulation purposes to speed up the ablation process of laser fiber 102. Catheter array 106 can be inserted into the central or lateral prostate lobes allowing thermal energy to be applied to a large portion of the prostate. Simultaneous combination of these two energy sources, one ablative, one coagulative, allows for an effective, safe and efficient removal of excess prostate issue thus contributing in great part to a successful BPH treatment.

In another preferred embodiment as shown in FIG. 2, BPH treatment device 200 consists of two or more laser energy sources emitting alternatively or simultaneously within a specific range centered at an absorption peak for specific tissue components. Output wavelength may be varied within a specified range and may be emitted in continuous, semi-continuous or pulsed mode. At least one laser energy source 204 drives an optical fiber 202 for transurethral laser ablation treatment of the enlarged prostate whose object is to vaporize a portion of the prostate tissue so as to allow for urethral flow. Another laser energy source drives a coagulating optical fiber 206, which comprises fiber body 212 and diffusing light fiber tip 210, used for interstitial access to the prostate. Special optical fiber 206 is introduced into a prostate lobe and provides diffuse laser power (due to diffusing light fiber tip 210) in order to necrose surrounding tissue. It can be inserted into the central or lateral prostate lobes by inserting a needle and a trocar transperineally into the middle of the lateral lobe, removing the trocar and inserting special optical fiber 206. One preferred wavelength range is 1950±50 nm. Preferred wavelength range presents a peak in water absorption and relatively very low absorption by other components present in tissues. Therefore, small variations in wavelength, within a small range modify tissue absorption behavior substantially. Physician can profit from this feature by varying emitting wavelength according to penetration needed and desired effects of ablation, coagulation or a combination of both on target tissue. In another preferred embodiment wavelength range is 1470+/−60. Additionally, by combining waveforms appropriately, using continuous wave or pulsed wave configurations, ideal simultaneous ablation/coagulation effects can be achieved. Fibers 202 and 206 may be, but are not limited to, off-axis firing end fibers, side-firing fibers, conical fibers, bare fibers, and radial emitting fibers.

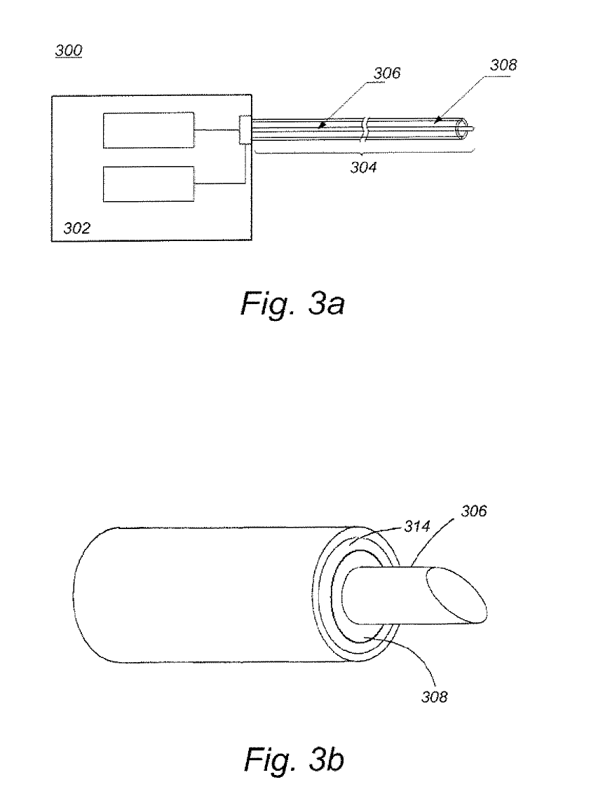

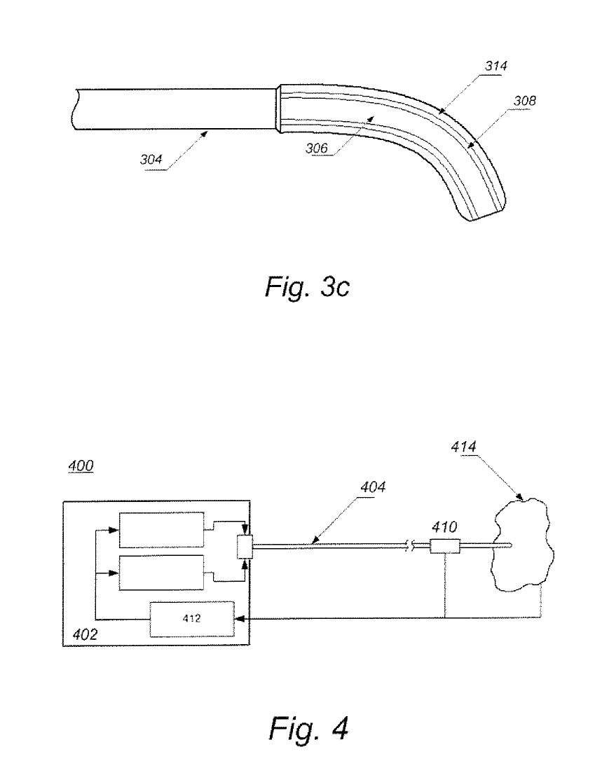

FIG. 3a depicts another preferred embodiment in which device 300 delivers alternatively or simultaneously two or more wavelengths to tissue through unique optical fiber 304. Optical fiber 304 is composed of a core 306 and two concentric claddings 308 and 314. Fiber Core 306 and outer cladding each conveys specific wavelengths. Inner cladding 308 must have a refraction index inferior to both core 306 and external cladding 314 so total internal reflection occurs and radiation is transmitted. For instance, wavelength combinations that may be used are 980±20 nm with 1950±50 nm and 980+/−20 with 1470+/−60 nm, with a range of power options to select from, to treat enlarged prostate or other abnormal tissue. Delivery of laser energy in the form of dual wavelength combinations is an efficient way to treat enlarged prostates and minimizing side effects. For example, it decreases the power necessary to treat the prostate and minimizes the probability of side effects to overlying skin and surrounding tissue. Laser energy can be delivered in pulse, Q-switched, semi-continuous and continuous mode. In a preferred embodiment, physician can adapt treatment by varying emitting wavelengths within a determined range according to penetration needed and desired effects of ablation, coagulation or a combination of both on target tissue.

FIG. 3b shows a detailed view of fiber tip whereby optical fiber 304 is composed of a core 306 and two concentric claddings 308 and 314. Fiber Core 306 and outer cladding each conveys specific wavelengths. Inner cladding 308 must have a refraction index inferior to both core 306 and external cladding 314 so total internal reflection occurs and radiation is transmitted. This particular embodiment describes a side emitting fiber (protective cap not shown).

FIG. 3c shows a close up view of fiber tip of another embodiment in which laser fiber has an off-axis firing end such as that disclosed in application Ser. No. 12/714,155 by Neuberger. Laser fiber 304 is composed of a core 306 and two concentric claddings 308 and 314. Fiber Core 306 and outer cladding each conveys specific wavelengths. Inner cladding 308 must have a refraction index inferior to both core 306 and external cladding 314 so total internal reflection occurs and radiation is transmitted. Other types of fibers, such as conical fibers, bare fibers, radial emitting fibers, etc. may be used for emission of more than one simultaneous wavelength.

In another embodiment, schematized in FIG. 4, device 400 comprises control mechanism 412, which allows for the delivery of constant power density based on feedback regarding speed of fiber 404 movement and local structural tissue 414 parameters. Handpiece 410 measures the speed of movement of optical fiber 404 and relays the feedback to control unit 412 which then controls the power output from laser surgery device 402. Laser surgery device 402 may have two or more lasers sources of appropriate wavelengths incorporated therein and connected to control unit 412. Additionally, laser surgery device 402 can house a motorized pull back device which can help to uniformly withdraw optical fiber 404 for use in certain treatments. This technique is an effective treatment for BPH, as the treated enlarged prostate is targeted with one or more laser radiation wavelengths. Treatment conditions are based on feedback from different control parameters that are monitored and previously determined structural parameters of the treatment site. Parameters such as movement speed, prostate size and temperature can be used to control the power to be delivered. According to these parameters, power density is automatically controlled by the device. This mechanism presents important advantages to the procedure. For example, it reduces over or under treatment, human errors and optimizes treatment parameters (power, pulse duration (T_{ON}), and pause (T_{OFF})). In another embodiment, control mechanism can also calculate appropriate emitting wavelength and choose between continuous, semi continuous and pulsed wave radiation according to measured parameters and desired treatment effect. Pulsed wave parameters may include pulse duration (T_{ON}), pause (T_{OFF}) and frequency.

The present invention is further illustrated by the following example, but is not limited thereby.

### EXAMPLE

In accordance with present invention, a medical device comprising a laser radiation source, for the generation of an ablative laser radiation source, at a variable wavelength of 1950±50 nm, feeds a fiber with a side-firing distal end (an optical fiber in which laser radiation is emitted perpendicularly to the longitudinal axis of the fiber, due to its tip configuration) for ablative purposes. Furthermore, several interstitial treatment members are attached to a microwave coagulative radiation source for coagulative purposes. In a variation of the mentioned example, a fiber with an off-axis firing end (an optical bent tip fiber with a fused cap as an integral part of it, placed at its distal end and with a rotatable connector at the proximal side) is used for ablation. This is useful when special steering, twisting and rotating movements are needed for a more precise an improved effect on target tissue.

At the beginning of the BPH treatment, interstitial coagulative probes are placed and left in place into the central or lateral prostate lobes allowing thermal energy to be applied to a large portion of the prostate. Once the placement of interstitial probes is done, ablative fiber is inserted transurethrally through a cystoscope. This ablative fiber can be moved and manipulated by the surgeon during the treatment to assure tissue removal in the critical locations and provide fast symptom relief. Physician may vary wavelength within a range of ±50 nm according results observed and results desired. While ablation process is occurring, coagulative probes produce the coagulation of underlying tissues to substantially eliminate blood loss beyond the removed tissue, with minimal thermal damage to surrounding tissue.

In the embodiments described, interstitial power reduces the volume of the prostate by inducing coagulation necrosis in the interior of the prostate.

Simultaneously or alternatively, the ablating optical fiber vaporizes prostatic tissue which, in some cases, better absorbs laser energy due to the coagulation process described. Thus, tissue coagulation can speed up the ablation process, in cases where necrosed tissue is easier vaporized by ablation laser energy.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A medical energy treatment device to carry out a Benign Prostate Hyperplasia treatment method, the device comprising:
at least one energy source capable of producing energy to coagulate tissue;
at least one laser source capable of producing radiation at a preselected wavelength, a power level and a power density to ablate tissue; and
at least one optical fiber optically coupled to said laser source and capable of transmitting said ablating radiation from said laser source to said target tissue.

2. The medical energy treatment device according to claim 1, wherein said energy source capable of producing energy to coagulate tissue is selected from the group consisting of a microwave source, a radiofrequency source, a laser and a thermal heat source.

3. The medical energy treatment device according to claim 2, wherein said coagulating energy source is preferably a non-laser energy source

4. The medical energy treatment device of claim 1, wherein said optical fiber comprises a distal end selected from a group consisting of a side-firing distal end, a radial firing end, and an off-axis firing end.

5. The medical energy treatment device of claim 1, further comprising a fiber optic probe for emitting said ablating radiation and a probe to transfer energy from said non-laser source to said target tissue.

6. The medical energy treatment device of claim 5, wherein said fiber optic probe comprises a termination selected from the group consisting of a bare fiber, a capped fiber, a fiber with a shaped end, a fiber with an off axis end, and combinations of these.

7. The medical energy treatment device of claim 1, wherein said given wavelength is selected from the group of 1470+60 nm and 1950±50 nm.

8. The medical energy treatment device of claim 1, wherein said at least one optical fiber has a core diameter of at least 200 μm.

9. A device comprising of an energy source capable of emitting both ablative laser energy as well as energy for coagulative purposes for the use in urology procedures and transmitting means capable of receiving energy from each type of energy device and transmitting said energy to a treatment site.

10. The device according to claim 9, wherein said energy source for coagulative purposes is a microwave source, a radiofrequency source, a laser or a thermal heat source.

11. The medical energy treatment device of claim 9, further comprising a motorized pullback device wherein pullback speed is based on feedback parameters received from said coagulation and ablation energy.

12. The medical energy treatment device of claim 1, wherein said at least one optical fiber comprises a core, an inner cladding and an outer cladding, each conveying specific wavelengths.

13. The device according to claim 12, wherein said inner cladding has a refraction index inferior to said core and said external cladding.

14. The device according to claim 4 wherein said optical fiber is composed of a core and two concentric claddings, an inner and an outer cladding respectively; said fiber core and said inner cladding each convey specific wavelengths; said inner cladding has a refraction index in between that of said core and said outer cladding.

15. A method of performing both a coagulative as well as an ablative Benign Prostate Hyperplasia (BPH) treatment in one session utilizing two types of delivery systems: a coagulative type that is essentially placed and left in place giving a sufficient time to achieve sufficient coagulation and an ablative device that can be moved and manipulated by the surgeon during the treatment to assure tissue removal in the critical locations and to provide fast symptom relief.

16. The method of BPH treatment according claim 15, comprising the steps of:
a. introducing a system for delivering energy to coagulate tissue at a treatment site;
b. activating said coagulative system and coagulating tissue at said treatment site;
c. introducing an ablative device, a probe for transmitting laser energy to a treatment site to ablate hyperplasic tissue;
d. activating said ablative laser system to ablate hyperplasic tissue with said probe;
e. ablating preselected hyperplasic tissue in a pulsed or continuous mode of applying said laser radiation;
f. positioning and repositioning said ablative laser energy probe, as required to ablate (remove) all desired hyperplasic tissue; and
wherein steps b. and c. can be interchanged in time sequence.

17. The method of BPH treatment according to claim 16, wherein said step f. is replaced by:
f. positioning said ablative laser energy probe at said treatment site; and
g. employing a motorized pullback device to withdraw probes, wherein pullback speed is based on feedback parameters received from said coagulation and ablation energy systems.

18. The method of BPH treatment according to claim 16, wherein said step of coagulating tissue employs a non-laser energy source.
